(19) United States
(12) Patent Application Publication
(10) Pub. No.: US 2014/0378778 A1
(43) Pub. Date: Dec. 25, 2014

(54) DYNAMIC MEDICAL ECOSYSTEMS MODELING

(71) Applicants: Camille HODGES, Youngsville, LA (US); Daniel HODGES, Youngsville, LA (US)

(72) Inventors: Camille HODGES, Youngsville, LA (US); Daniel HODGES, Youngsville, LA (US)

(21) Appl. No.: 14/212,250

(22) Filed: Mar. 14, 2014

Related U.S. Application Data
(63) Continuation-in-part of application No. 14/205,844, filed on Mar. 12, 2014.
(60) Provisional application No. 61/783,996, filed on Mar. 14, 2013.
(60) Provisional application No. 61/950,318, filed on Mar. 10, 2014.
(60) Provisional application No. 61/934,090, filed on Jan. 31, 2014.

(51) Int. Cl. A61B 5/00; A61B 5/11

(52) U.S. Cl. CPC A61B 5/7275; A61B 5/0022; A61B 5/4857; A61B 5/1112; A61B 5/1118; A61B 5/6847
USPC 600/301

(57) ABSTRACT

Systems and methods of embodiments comprise receiving physiological data that includes data of multiple physiological parameters collected in real-time via sensors coupled to an individual entity. Micro plots are generated, and each micro plot comprises a cyclical plot of the physiological data for a corresponding time period of a multitude of time periods. A medical model plot is generated to include the micro plots. Formation of the medical model plot involves plotting of the micro plots chronologically according to the time periods. A location of an endpoint of each micro plot determines a change in slope of the medical model plot, and the slope represents a state of health of the individual entity.

### RELATED APPLICATIONS

This application claims the benefit of U.S. Patent Application No. 61/783,996, filed Mar. 14, 2013.

This application claims the benefit of U.S. Patent Application No. 61/950,318, filed Mar. 10, 2014.

This application claims the benefit of U.S. Patent Application No. 61/934,090, filed Jan. 31, 2014.

This application is a continuation in part of U.S. patent application Ser. No. 14/205,844, filed Mar. 12, 2014.

### TECHNICAL FIELD

The embodiments described herein relate generally to systems and methods for modeling and, more particularly, to dynamic medical ecosystems modeling.

### BACKGROUND

The basis for organized medicine was established in approximately 400 B.C. Since then the art and practice has essentially been one of single point probabilistic approximation and formulation. For over two-millennia brief encounters with the treating physician or their staff has represented the pillar of established medical practice and healthcare delivery. Medicine as a discipline in the 21st century clearly has had the advantage of exponential growth in healthcare technology particularly over the past thirty years, but at its very core, the physician's single point probabilistic approximation and formulation remain (differential diagnosis) all but unchanged in its 2500-year existence. There is a need for micronization of the future of medicine under a new paradigm that promises to revolutionize the practice and delivery of healthcare.

### INCORPORATION BY REFERENCE

Each patent, patent application, and/or publication mentioned in this specification is herein incorporated by reference in its entirety to the same extent as if each individual patent, patent application, and/or publication was specifically and individually indicated to be incorporated by reference.

### BRIEF DESCRIPTION OF THE DRAWINGS

FIG. 1 is a block diagram of an example of static moments in patient treatment practice.

FIG. 2 is a plot of the Life Cycle Line, under an embodiment.

FIG. 3 is a completed graphical representation of a Life Cycle Line of a first individual, under an embodiment.

FIG. 4 is a completed graphical representation (post-mortem) of morbid childhood obesity and poor impulse control that has a snowballing effect on the individual's health and wellness, under an embodiment.

FIG. 5 is a completed graphical representation of a Life Cycle Line where the individual is in the normal zone, and was subject to random environmental factors, under an embodiment.

FIG. 6 is a block diagram showing development of the dynamic Medical Ecosystems Model (dMEM), under an embodiment.

FIG. 7 shows a flow diagram of Life Cycle Line development, under an embodiment.

FIG. 8 shows slope changes in the dMEM, under an embodiment.

FIG. 9 shows the dynamic Life Cycle Line, under an embodiment.

FIG. 10 shows a plot of a Life Cycle Line depicting an individual who did not incorporate any kind of health monitoring into their lives, an Unmonitored Lifestyle, under an embodiment.

FIG. 11 shows a plot of a Life Cycle Line depicting a Monitored Lifestyle, under an embodiment.

FIG. 12 is an example dMEM recording of the first patient, under an embodiment.

FIG. 13 is an example dMEM recording of the second patient, under an embodiment.

FIG. 14 shows the dMEM recording of the previous seven-day compressed data compilation of this patient's pain pattern, under an embodiment.

FIG. 15 is a plot of the cure rate of the patient having only C-spine surgery, under an embodiment.

FIG. 16 is a plot of the cure rate of the patient having C-spine and carpel tunnel release surgeries, under an embodiment.

FIG. 17 depicts the superimposed similarity between the pain from carpal tunnel syndrome (CTS), and recent onset cervical radiculopathy, under an embodiment.

FIG. 18 illustrates multiple end users linked to the dMEM cloud, under an embodiment.

FIG. 19 is a block diagram of the dMEM integrated with a supercomputer system, under an embodiment.

FIG. 20 depicts a helicoid example underlying the dMEM system design, under an embodiment.

FIG. 21 is a block diagram depicting a dMEMs platform hosting the circadian model, under an embodiment.

FIG. 22 is a block diagram depicting the dMEMs platform creating a real-time sensing and collecting system running in parallel to human physiology, under an embodiment.

FIG. 23 shows the dMEMs from the perspective of the application/nano-sensor developer, under an embodiment.

FIG. 24 shows the dMEMs from the perspective of the public end-user, under an embodiment.

FIG. 25 shows the dMEMs from the perspective of the active practice physician, under an embodiment.

FIG. 26 shows an example of the dMEMs from the perspective of the active practice physician when treating a patient following patient discharge, under an embodiment.

FIG. 27 shows an example of the dMEMs from the perspective of the active practice physician when treating critical care patient, under an embodiment.

FIG. 28 is a block diagram of the dMEM integrated with medical smart systems, under an embodiment.

### DETAILED DESCRIPTION

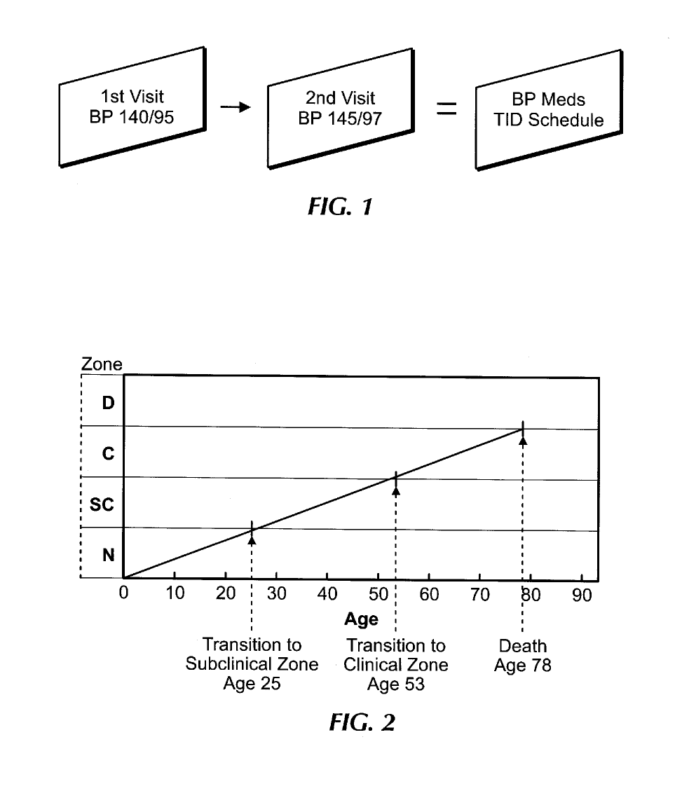

The conventional practice of medicine has been one of static formulation. Static moments or points in time (much like a “snapshot” or photo) have been the basis for practicing modern medicine. For example, a physician may check a patient's blood pressure on two different occasions (office visits) over a two-week period. FIG. 1 is a block diagram of an example of static moments in patient treatment practice. The snapshot of information obtained during the two visits is then used to determine and initiate a medication regime with an antihypertensive agent. For example, a symptomatic 44-year-old overweight male patient complaining of a two-week history of fatigue and dizziness newly presents to a family physician's office at 4 pm on a Friday afternoon. His blood pressure as checked by the office nurse is noted to be marginally elevated and his screening blood profile normal. He is told to follow up in the family physician's office in two weeks. On the second visit the patient's blood pressure remains elevated. This brief momentary dual snapshot of information obtained during two separate visits at different times, and under different conditions, is then used by the physician to access and briefly determine a differential diagnosis as to the probable etiology of the patient's High Blood Pressure. Then the physician will initiate what he or she may deem to be an appropriate medication regime, i.e. with an antihypertensive agent three times per day.

Conventional medical treatment and diagnosis therefore leaves much to be desired in terms of efficacy when one is establishing a diagnosis based upon two brief office visits separated by a 14-day interval. Furthermore inefficiency becomes evident, if not obvious, in an antiquated medication delivery system based on a one-size fits all approach (i.e. TID schedule). When in reality, diurnal morning and evening blood pressure spikes may well have caused an inaccurate diagnosis. The troubling consequences of this, include this one-time walk-in patient now on the street, suffering from potentially life-threatening rebound iatrogenic medication induced hypotension.

Embodiments described herein include a medical modeling system or platform, also referred to as the “dynamic Medical Ecosystems Model” (dMEM), that redefines the practice of medicine through proprietary processes of real-time dynamic medicine incorporating nano-sensors. The dMEM creates and applies to individuals a novel real-time health continuum, where generation and application can begin at the moment of birth. The dMEM monitors an individual's health throughout their life. As such, the dMEM provides a virtual platform, creating and enabling preemptive-preventive self-care delivery in real-time. The dMEM along with technological advances in medical nano-sensors will drive the novel medical paradigm, forever changing the scope and practice of human healthcare.

Although the detailed description herein contains many specifics for the purposes of illustration, anyone of ordinary skill in the art will appreciate that many variations and alterations to the following details are within the scope of the embodiments described herein. Thus, the following illustrative embodiments are set forth without any loss of generality to, and without imposing limitations upon, the claimed invention.

The dMEM of an embodiment develops a Life Cycle Line model, or static life cycle, into a dynamic living model as described in detail herein. FIG. 2 is a plot of the Life Cycle Line, under an embodiment. In essence, a completed (static) Life cycle Line represents the linear graph of an individual's cumulative life, beginning at birth, progressing through normal, subclinical, and clinical zones culminating in death. In the completed cycle, the slope of an individual's Life Cycle Line may have increased or decreased as a result of life choices. The graphic indicates the male in this case, had a relatively disease free life. At the age of 25 he crossed from the normal zone N (no underlying disease process) into the subclinical zone SC (no symptoms to indicate disease, but detectable disease is apparent on labs, imaging studies, etc.; statistically, the majority of the US population enters a subclinical disease zone as early as the third decade with the onset of clinical symptoms by the fifth decade), and by age 53 years a low-grade clinical zone C (disease state arriving to the point of symptom presentation causing the patient to seek medical attention; this is currently the main point of entry into US healthcare by most first-time patients). From age 53 years to 78 years his symptoms, in the clinical zone gradually progressed upwards with death D occurring at 78. The Life Cycle Line ends upon the death of the corresponding individual.

The upward or downward slope of an ongoing dynamic Life Cycle Line determines not just the length of one's life but the quality as well. It is anticipated that much of twenty-first century healthcare will be directed towards assisting individuals in the proper day-to-day data management of his or her dMEM on the dynamic Life Cycle Line model. In doing so, metabolic disease states, both chronic and acute, will be eradicated with marked overall reductions in early life morbidity, disability, and death. It is presently known that well over 45% of progressive acute and chronic metabolic disease states in the U.S. are due to early-learned aberrant behavioral patterns. Learned behavioral patterns are subject to modification and can be positively modified by the dMEM, resulting in dramatic improvements in overall lifelong health and wellbeing.

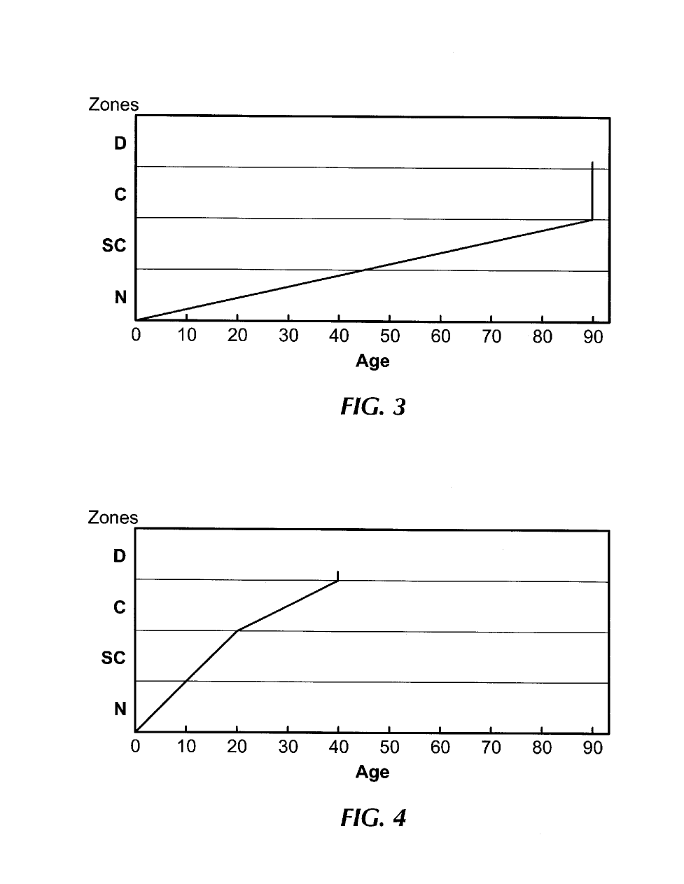

Detailed examples are presented below to illustrate how the Life Cycle Line may appear in three different people, with three distinct causes of death. FIG. 3 is a completed graphical representation of a Life Cycle Line of a first individual, under an embodiment. The individual of this example has properly cared for their health and wellness, living to an age of 90 years with great quality of life, under an embodiment. Even in an individual who is healthy, the natural course of events range from normal at birth, with varying transitions into sub-clinical and clinical disease prior to death. The 90-year-old individual maintained a great quality of life, without significant lifetime clinical (symptomatic) disease, as per her Life Cycle Line. Although subclinical (asymptomatic) disease was evident from the age of 45 years until the time of her demise, death in this case occurred due to an unexpected head-trauma from a fall resulting in a massive hemorrhagic stoke.

FIG. 4 is a completed graphical representation (post-mortem) of morbid childhood obesity and poor impulse control that has a snowballing effect on the individual's health and wellness, under an embodiment. At age ten years, the individual crosses into the subclinical zone (elevated blood sugars), eventually crossing into the clinical zone by age 20 with adult onset insulin dependent diabetes. The ability to directly quantify a person's everyday actions into one output is a key concept in the Life Cycle Line.

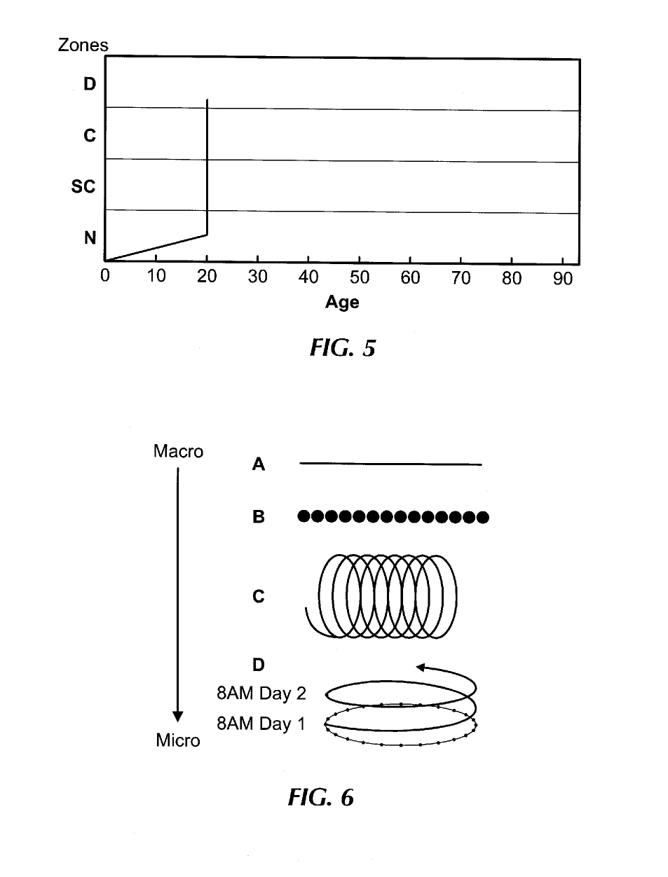

FIG. 5 is a completed graphical representation of a Life Cycle Line where the individual is in the normal zone, and was subject to random environmental factors, under an embodiment. His Life Cycle Line went from the normal zone vertically through subclinical and clinical zones into death. He was a healthy twenty-year old who was originally projected to live for at least 83 years. This individual's life was cut drastically short, killed in action in Afghanistan at age 20.

Consideration of the linear graphical components described herein provides context for the more complex underlying processes of the dMEM of an embodiment. For the sake of the description herein, the following example will employ a hundred year life. FIG. 6 is a block diagram showing development of the dMEM, under an embodiment. A hundred years on the sloping lifeline in this model will equal 36,500 days of life, representing a single Life Cycle Line A (continuous uninterrupted life cycle line) of 36,500 connecting points B (composite points that comprise continuous line A). Hence a single point on the Life Cycle Line represents one day or a completed 24-hour cycle, which equates to a 360-degree circle. It is upon this circle the nano-monitored events or physiological data of the previous 24-hour period is recorded and analyzed. The 24-hour event cycle is in reality a continuous helix in the dMEM process as described in detail herein. It returns to the same beginning point every 24 hours, but due to the passing of time it is located at a slightly different point in space.

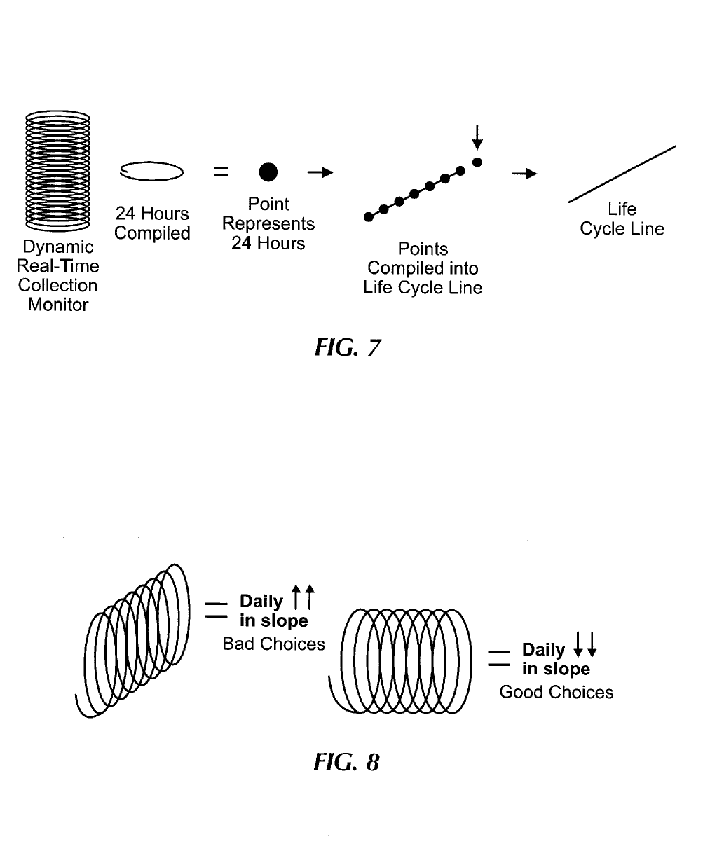

With reference to FIG. 6, the basis for the dMEM process model or computer model arises from C (each completed cycle on the helix represents a composite point on B) and D (each mapped 24-hour cycle (e.g., 8 am to 8 am) represents a composite revolution on C) in the diagram showing dMEM development. The dMEM of an embodiment includes a 24-hour cycling processor-based (e.g., server, cloud, personal computer, etc.) platform that collects real-time multiples of physiologic data from medical micro sensors (external or internal), while using the cyclic models of C and D, to essentially change the Life Cycle Line from a static to a dynamic entity. The helical recordings of each 24-hour cycle maintain all medical data corresponding to an individual. Daily 24-hour cyclic recordings can then be plotted to a patient's Life Cycle Line. FIG. 7 shows a flow diagram of Life Cycle Line development, under an embodiment.

More particularly, the dMEM receives physiological data that includes data of multiple physiological parameters collected in real-time from sensors coupled to an individual subject. The sensors are coupled to or implanted in the subject, and are configured to telemeter the physiological data to the dMEM platform or otherwise offload or download the physiological data to the dMEM platform. The sensors of an embodiment include sensors of any type and/or configuration as appropriate to collection of physiological data from a living entity. Furthermore, the physiological data includes any data or parameters capable of being collected from a human subject.

Upon receiving the physiological data, the dMEM generates a number of micro plots, where each micro plot represents or corresponds to a particular time period. Each micro plot includes a cyclical plot of the physiological data for a corresponding time period (e.g., 24-hour period, etc.). Thus, each micro plot comprises an integrated plot of all physiological data collected from a subject during the corresponding time period. Using the micro plots, the dMEM generates a medical model plot, or Life Cycle Line. In generating the medical model plot, the dMEM plots the micro plots chronologically according to the corresponding time periods, such that a location of an endpoint of each micro plot determines a change in slope of the medical model plot. As described in detail herein, the slope of the medical model plot represents a state of health of the human subject.

The successful dMEM from 0 to 90 years equals a connected series of 32,850 points representative of a continuum of days. Furthermore, each point may then be reduced to a non-random reoccurring 24-hour cycle that essentially returns to the same position after completion of a 360° rotation over 24 hours. Each hour represents 1/788, 400^{th} of 90 years and equates to 15° rotation on each 360° cycle. Each rotation returns to the same point in the 360° rotation but has moved in space to a new position representing the end of one 24-hour cycle and the beginning of a new 24-hour cycle. Hence the dMEM exists as a helical entity in space.

The point in space that ends each monitored cycle, determines the change of slope in the dMEM. This slope change correlates to the actions and behaviors of the preceding 24-hour cycle. The dMEM precisely displays changes in an individual's monitored actions and choices for the upcoming day, based upon the choices made the day before. FIG. 8 shows slope changes in the dMEM, under an embodiment. Representative drivers for an increased slope (less time in the normal zone, shorter life, worse quality of life) include, but are not limited to, the following: aberrant behavioral patterns; excessive alcohol consumption; unhealthy, unbalanced diet; tobacco use; sedentary lifestyle; depression; low education levels; living at or below the poverty level. Representative drivers for a decreased slope (more time in normal range, longer life, better quality of life) include, but are not limited to, the following: no alcohol consumption; healthy balanced caloric diet; no tobacco use; active lifestyle; good mental hygiene; higher education levels; living above poverty level.

Therefore, the plotting of each 24-hour cycle of monitored physical and metabolic parameters and changes produces a continuous helix representing the true dynamic nature of the dMEM. By dissecting the helix, each previous and succeeding monitored cycle can be directly compared with others. For example, once a baseline of seven consecutive cycles is obtained, these may be sequentially compressed to a single cycle, yielding a weekly compilation. Uniform compression of a month, year, or decade becomes possible as aging data becomes available for compression. The end-user will have multi-sourced feedback available continuously.

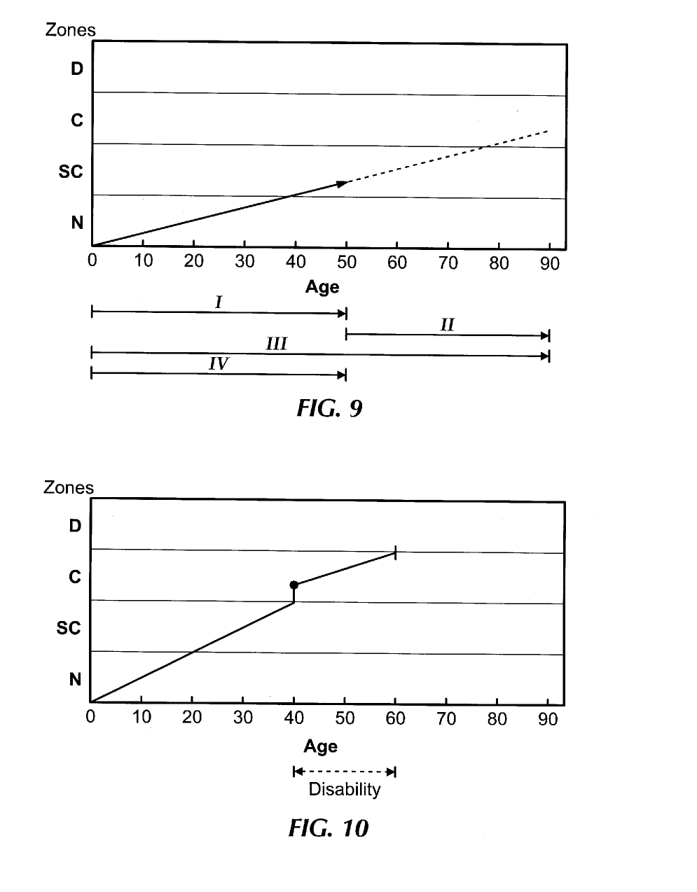

New medical paradigms are emerging of which the dMEM will be a major component. For example, a first medical paradigm is one in which preemptive “self-healthcare” will virtually eradicate acute and chronic disease states. A second medical paradigm is one that reveals previously undiagnosed disease, significantly augmenting future medical and surgical outcomes. A third medical paradigm is one in which future dynamic medical, biomedical, pharmacological, academic, and epidemiologic research and stratification, changes the face of global health. A fourth medical paradigm is one in which supercomputers, physiology, and medicine become a singular dynamic real-time continuum. FIG. 9 shows the dynamic Life Cycle Line, under an embodiment. The Life Cycle Line demonstrates the future point of entry, integration, and flow of the emerging paradigms (I, II, III, IV) in the healthcare continuum. A detailed description of the new medical paradigms follows.

With reference to the first new medical paradigm, an example of the dMEM as a preemptive “self-healthcare” model can be demonstrated by comparison of the following completed Life Cycle Lines. The dMEM of an embodiment virtually eradicates acute and chronic disease states. FIG. 10 shows a plot of a Life Cycle Line depicting an individual who did not incorporate any kind of health monitoring into their lives, an Unmonitored Lifestyle, under an embodiment. Thus, they did not have feedback regarding how their day-to-day choices truly impacted their future health and longevity. This individual suffered from a massive heart attack at age 40 years due to his aberrant behavioral patterns. He survived the event, but as noted in the Life Cycle Line, from age 40 years until death at age 60 years, the patient remained permanently and totally disabled, dependent upon government resources.

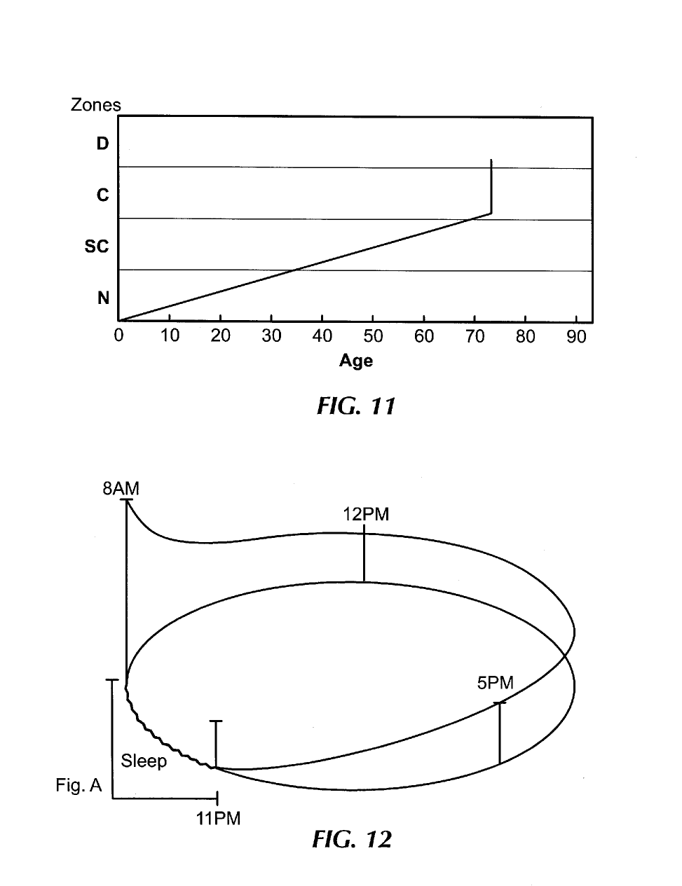

FIG. 11 shows a plot of a Life Cycle Line depicting a Monitored Lifestyle, under an embodiment. This individual's health was monitored from age 10 years. The data collected continuously from the individual was processed via the dMEM to yield his Life Cycle Line. From the time this individual was a child, he and his parents had the benefit of knowing how his (and his parents′) choices were impacting him. The child would learn from a much earlier age which choices in his life are truly healthy. The visual feedback from the Life Cycle Line would provide positive reinforcement for healthful living from a very young age. This preemptive “self-healthcare” would necessarily eliminate 45% of debilitating acute and chronic metabolic disease states in the U.S. Healthcare dollar savings would be tremendous. With the Monitored Lifestyle, the patient was able to see an improved quality of life, improved longevity, and productive lifestyle with absence of disability, until death at 74.

Under the second new medical paradigm, in the diagnosis and treatment of existing acute and chronic disease states, a shift occurs from preemptive preventive medicine, to one of treatment of established disease, as osteoarthritis, rheumatoid arthritis, carpal tunnel syndrome, and cervical radiculopathy are explored.

In a first example under the second new medical paradigm, two 60-year-old male patients, new to the doctor's office on the same day, complain of generalized aches and pains consistent with arthritis. The tendency is to treat them medically based on a “snapshot moment in time” office visit. In this case they both would likely be treated symptomatically with anti-inflammatory medication and sent home. When the monitored cyclic dMEM is applied on a real-time 24-hour cycle for seven days and then compressed, two distinctly different patterns of pain begin to emerge. The first patient would elicit a pattern analogous to rheumatoid arthritis with progressive pain at its zenith in the early morning hours, as seen in the patient's dMEM recording. FIG. 12 is an example dMEM recording of the first patient, under an embodiment.

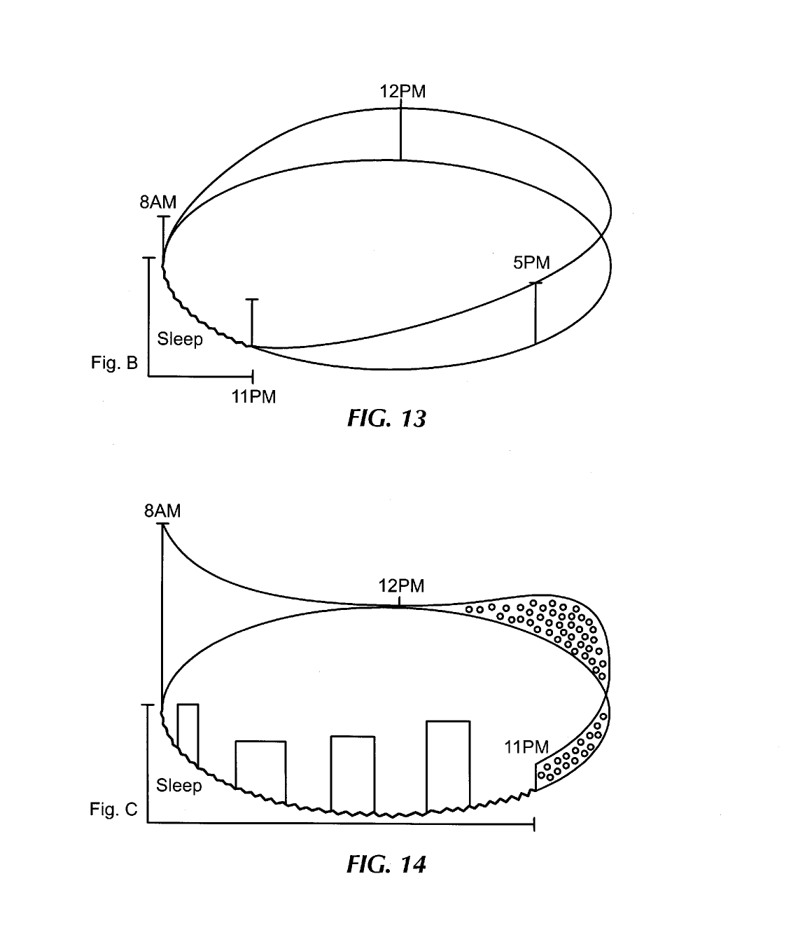

The second patient's pain pattern will demonstrate its true zenith in the mid afternoon, which would indicate degenerative arthritis, as seen in the patient's dMEM recording. FIG. 13 is an example dMEM recording of the second patient, under an embodiment. This becomes apparent in the real-time recurrent cycling “movie” while not recognized nor likely considered by modern day “snapshot” medicine. Both patients are misdiagnosed as a result, and neither receives accurate or appropriate care.

The relative differences seen between the dMEM recording of the seven-day compressed data compilation (FIG. 12) of a pain pattern in the first patient with rheumatoid arthritis, and the dMEM recording of the seven-day compressed data compilation (FIG. 13) of a pain pattern of the second patient with degenerative arthritis shows that the treatments for active rheumatoid arthritis are vastly different than the prescription for anti-inflammatories the patient was given. With the use of dMEM monitoring, the previously unseen physician errors, misdiagnoses, and inappropriate treatments are quickly revealed.

A 45-year-old patient presenting with radiating neck, arm, and hand pain represents a second example under the second new medical paradigm. He was recently involved in a motor vehicle accident (MVA) and has cervical radiculopathy, and is currently awaiting C-spine surgery in five days, as recommended by his neurosurgeon. The patient worked as a diesel mechanic, and had well documented pre-existing occasional hand pain radiating into digits 1, 2, 3, and forearm prior to the MVA. He now has severe hand pain running into digits 1, 2, 3, with forearm, arm, and neck pain. FIG. 14 shows the dMEM recording of the previous seven-day compressed data compilation of this patient's pain pattern, under an embodiment. The pain clearly varies during course of the day. The dotted elevation in pain is denoted as primarily sharp neck, shoulder, and forearm pain radiating into the hand. The solid red markers indicate dull pain occurring primarily in the hand and forearm.

The pain diagram, to an astute clinician using the dMEM, will be obvious and can be easily compared to stored database renderings to confirm the diagnosis, with a probability nearing one. The patient, in reality, has pre-existing low-grade carpel tunnel syndrome that is now acute secondary to the double crush from MVA induced acute cervical radiculopathy. The patient has two diagnoses and will need two surgeries: a C-spine surgery and carpel tunnel release before he will get total relief.

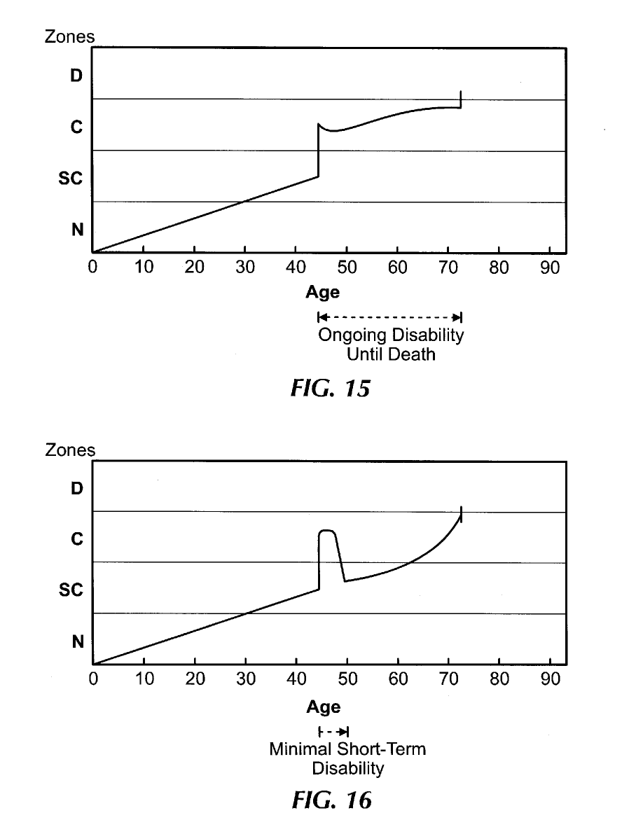

Without the dynamic dMEM compressions he would have likely been diagnosed and treated with C-spine surgery only. His cure rate would have been reduced to 33% with chronic pain and ongoing disability until death. FIG. 15 is a plot of the cure rate of the patient having only C-spine surgery, under an embodiment.

If both diagnoses had been made and both surgeries performed, his cure rate would have been 85%, with minimal short-term disability. FIG. 16 is a plot of the cure rate of the patient having C-spine and carpel tunnel release surgeries, under an embodiment.

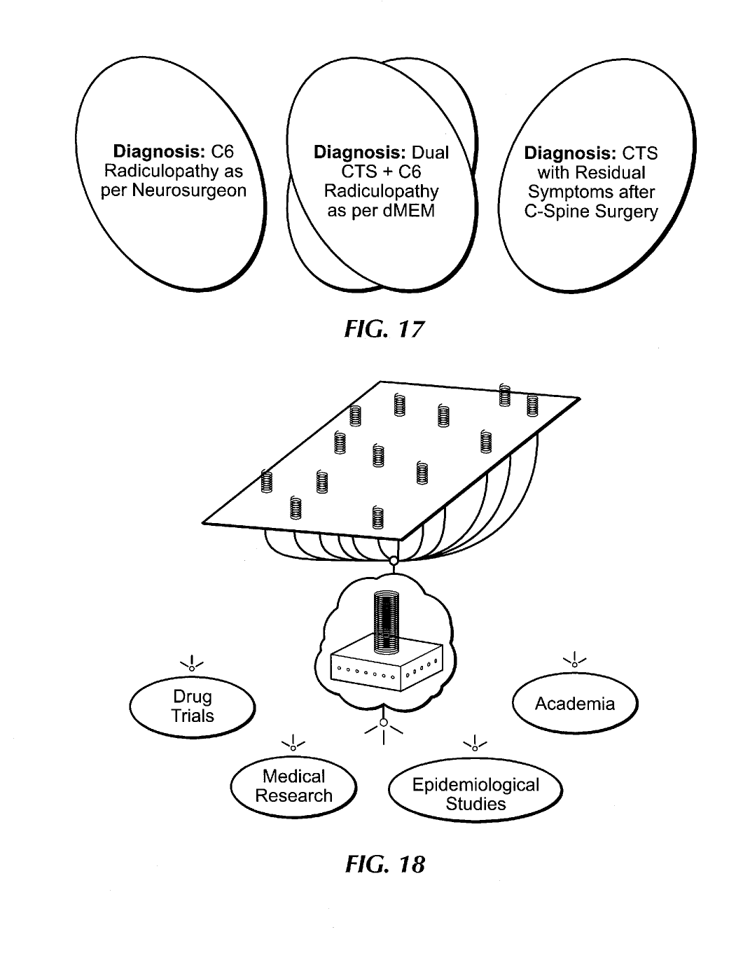

FIG. 17 depicts the superimposed similarity between the pain from carpal tunnel syndrome (CTS) (right-tilted oval), and recent onset cervical radiculopathy (C6) (left-tilted oval), under an embodiment. The C6 oval illustrates the treating physician's assumed single diagnosis based on MRI changes consistent with cervical radiculopathy, but further assessment via dMEM compressions would have clearly revealed a dual overlapping diagnosis (crossed ovals) of cervical radiculopathy C6 and secondary carpel tunnel syndrome (CTS), creating a double crush phenomenon.

Referring to the third new medical paradigm, FIG. 18 illustrates multiple end users linked to the dMEM cloud, under an embodiment. This allows for scalable real-time data acquisition. Multiple end user institutions (medical, academic, among others) under this embodiment select from a number of parameters they wish to monitor or study in real-time. For example, in the diagram below, the hypothetical plane may represent an institution's selected area of current study. This could include geographical distribution, age distribution, race distribution, disease prevalence, etc. All of these parameters and more are monitored in real time using the dMEM.

Drug companies, for example, will use the dMEM to monitor multiple cohorts of study participants in ongoing real-time clinical trials. This will undoubtedly change the dynamic of clinical drug trials with the earliest yet recognition of a drug's efficacy, safety, as well as unanticipated positive or negative collateral side effects.

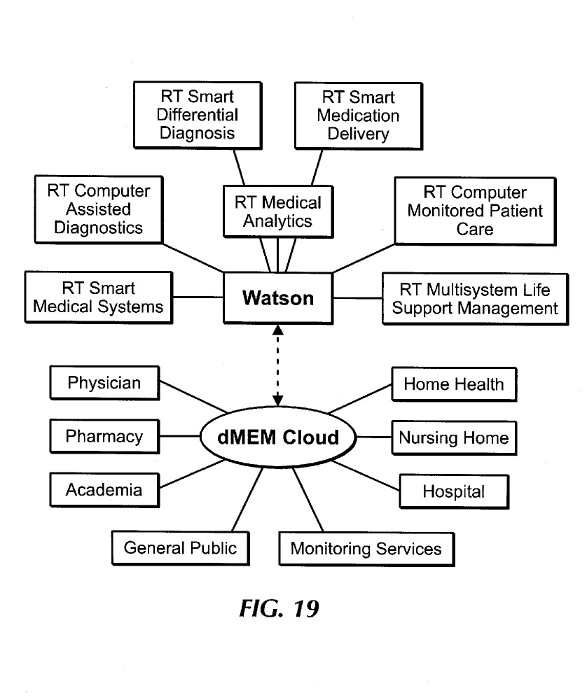

Referring to the fourth new medical paradigm, the integration of a supercomputer system into the dMEM ensures that every individual, patient, hospital, and medical institution in the world will have a continuous open-ended flow of real-time input and data collection from global supercomputer guided diagnostics and treatment. FIG. 19 is a block diagram of the dMEM integrated with a supercomputer system, under an embodiment.

In the past three decades, U.S. Healthcare has experienced exponential technological growth in “linear” diagnostic imaging and treatment systems. These advances have consistently been directed and ultimately designed to assess and or treat established pre-existing acute and chronic disease states, often as design-specific “post-event” diagnostic and treatment modalities i.e. heart, stroke, cancer care, etc.

For this reason, all diagnostics developed and introduced in the past thirty years tend to cluster around the after-the-fact points of clinical presentation due to chronic dysfunctional and acute event occurrence (heart attack). No significant preventive measures of any kind have been able to change the paradigm to this point in time. During this same thirty-year period, the medical dollar spent on design and development of preemptive preventable disease management lagged far behind. This was particularly true for the concept of medical ecosystems development until recent advancements in medical nano-sensors established a real and present niche-need. Nevertheless, it is calculated that a rapid paradigm shift to multi-dimensional medical ecosystem will significantly impact the anticipated growth curve in “linear” healthcare research and development over the next two decades. This will be particularly evident as the scalable medical ecosystems of the dMEM provide and guide individual health and healthcare delivery on a real-time ‘day to day’ basis by the application of its preemptive medical capabilities.

The state of medical nanotechnology is evolving rapidly, and the multiplicity of nano-sensor and sensor derivatives expected to enter the market over the course of the next five years will see exponential growth in numbers. Diversity of development, sensitivity and continued diminution in size will ensure that an expanding array of disparate technical and medical applications will continually be available to the mobile general public. However, in the coming years as industry maturity occurs, the surviving spectrum of segmented medical apps will be forced to unify and standardize across the board before nanotechnology as a emerging field in medicine may flourish.

The conventional novelty applications running at any one time measuring an individual's vital signs may suffice for the younger health-oriented segment of society. These applications, or apps, can be configured to continuously monitor for a “triggering event” such as an irregular heartbeat while an end-user is exercising. At that point where the anomaly is sensed, the data capture on the end user can increase, by initiating other apps (e.g., an app for cardiac enzymes) to monitor associated parameters.

That said, to become a fully integrated adjunct in the future of both preemptive-preventive and acute critical care medicine, the systems of the dMEM of an embodiment are configured to run and monitor 60 to 200 and more integrated real-time apps on an ongoing 24/7 basis. As a result, the dMEM makes use of a mammoth data collection-compression architecture with sensitivity extending well beyond linear and planar mappings of 24 hours. The computing hardware, storage and bandwidth for such an endeavor is readily available with cloud-based web-services and data-centers offered by third party providers. The limiting factors will not be computer or hardware capacities, but rather innovative configuration and integration. Medical nano-sensors combined with the dimensionality of real-time human physiology will push present computing architectures into a multi-dimensional framework.

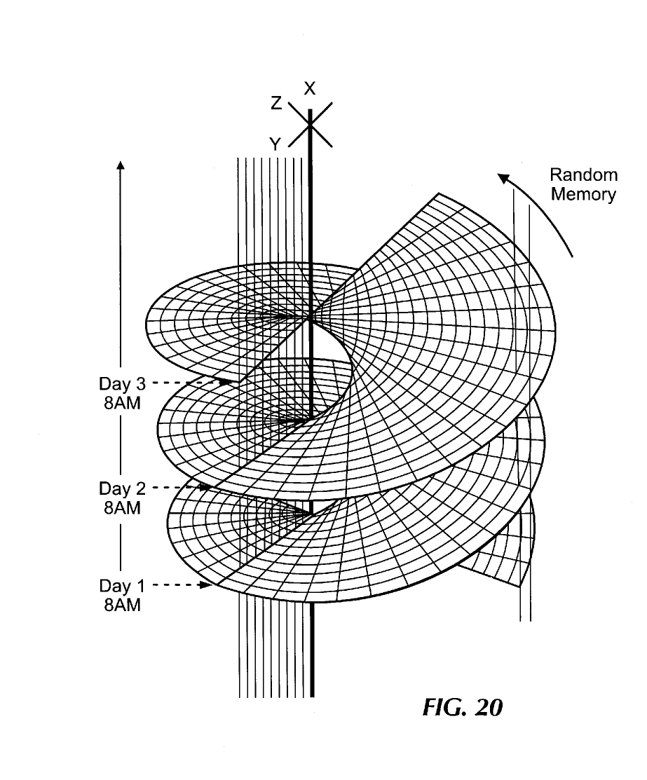

Since the beginning of time, intelligent life on earth has been dependent and unknowingly subservient to cyclic patterns (daily, monthly, yearly). The most obvious of these patterns is the 24-hour circadian cycle, established by earth's rotation. This perpetually reoccurring 24-hour cycle has had countless millions of years programming human life to respond and thrive upon a cycled existence. FIG. 20 depicts a helicoid example underlying the dMEM system design, under an embodiment.

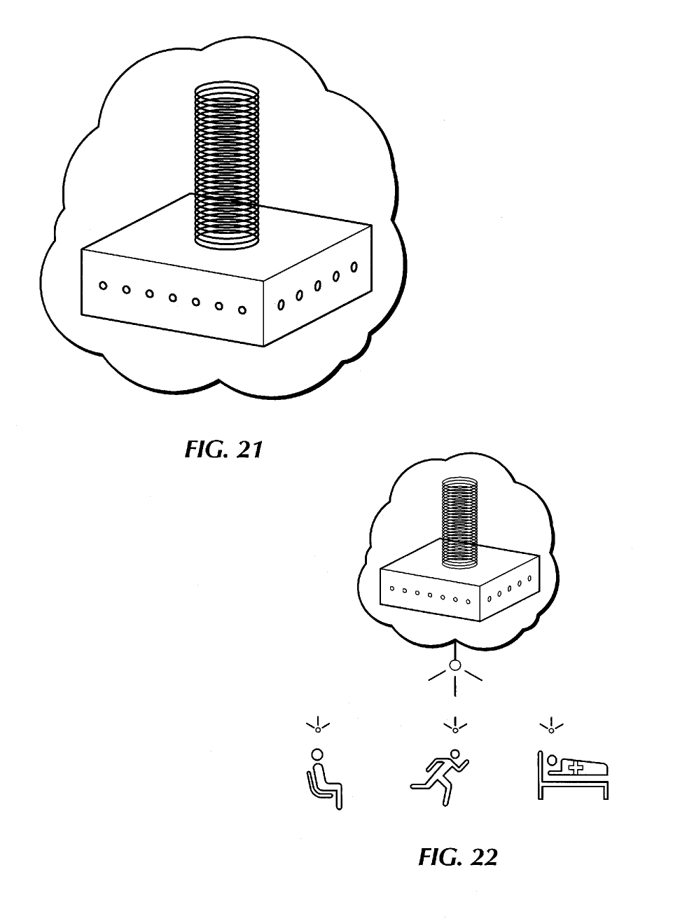

The architecture and running system of embodiments described herein give much attention and consideration to a three-dimensional (3D) composite world, to run parallel with real-time physiologic data capture in conjunction with person place time and event. FIG. 21 is a block diagram depicting a dMEMs platform hosting the circadian model, under an embodiment.

A cloud driven helical architecture is a paradigm changer for the future of medicine. FIG. 22 is a block diagram depicting the dMEMs platform creating a real-time sensing and collecting system running in parallel to human physiology, under an embodiment. Standardization of pre-configured plug and play ports to the cloud platform, the nano-sensor hardware developer need only configure sensor software to interface with the cloud's ports. Each medical nano-sensor developer and their respective software engineers will be provided hands-on tutorials and technical assistance to grasp a thorough understanding of the 3D real-time architecture and the 24/7 operating systems requirements.

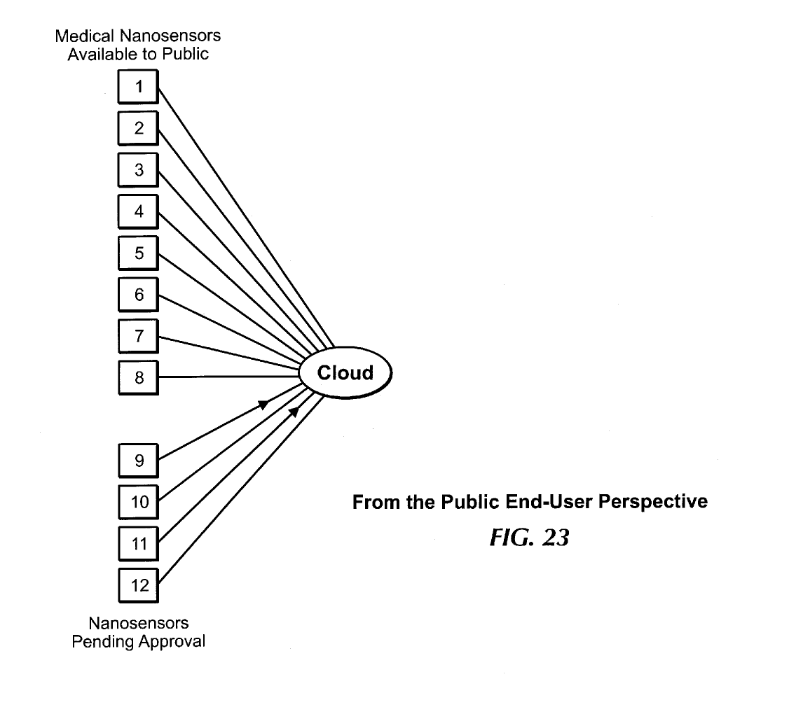

FIG. 23 shows the dMEMs from the perspective of the application/nano-sensor developer, under an embodiment. Approaching the embodiments described herein from the perspective of the application/nano-sensor developer, when the app/nano-sensor is approved and selected for port to real-time system migration, the accompanying nano-sensor/app becomes available on the cloud platform to be downloaded and applied to the end users handheld or tablet device. Each app/nano-sensor will be dormant on the cloud until an end-users interface is activated and usage begins.

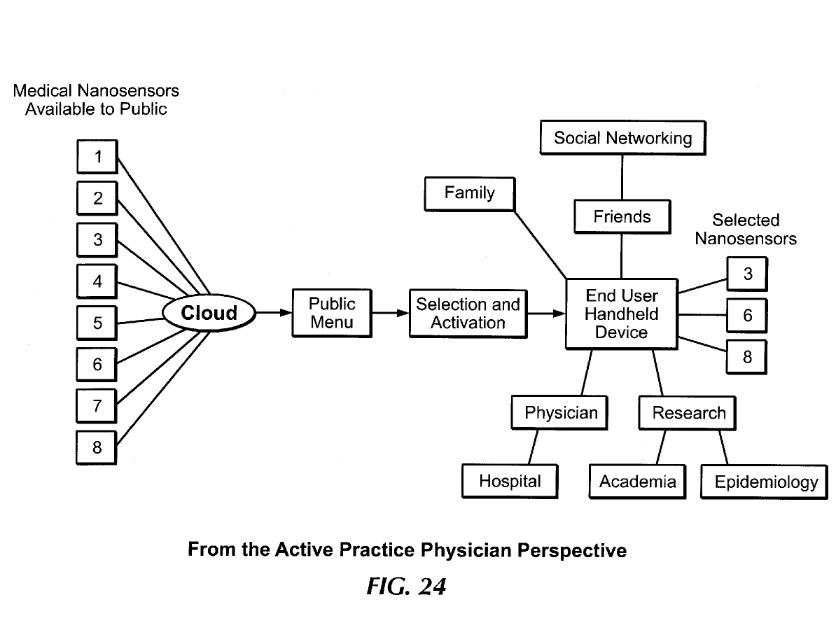

FIG. 24 shows the dMEMs from the perspective of the public end-user, under an embodiment. Approaching the embodiments described herein from the perspective of the public end-user, when a new cloud account in opened by an individual, he or she may then, depending upon credentialing be given access to select from approved app/nano-sensor that may be appropriate for public usage. These will be listed on the cloud-based open public interface, much like an app store. Each app will provide a detailed medically oriented description of available usage for the potential end-user, as well as bundling capabilities, bandwidth needs, ordering instructions for hardware, cloud fees, etc. The site owner may also select to provide viewing rights to other family members, various care providers such as physicians, nurses, home health providers, emergency services providers, hospitals and research institutions, etc. Categories of public self-tracking users include the young health conscious adult who wants daily tracking of basic vital health systems linked and plotted to the Life Cycle Line. Monitored users as a category, may be nursing home patients tethered to family physician, hospital, home health, family members, as well as yet to be created general and specialty monitoring systems.

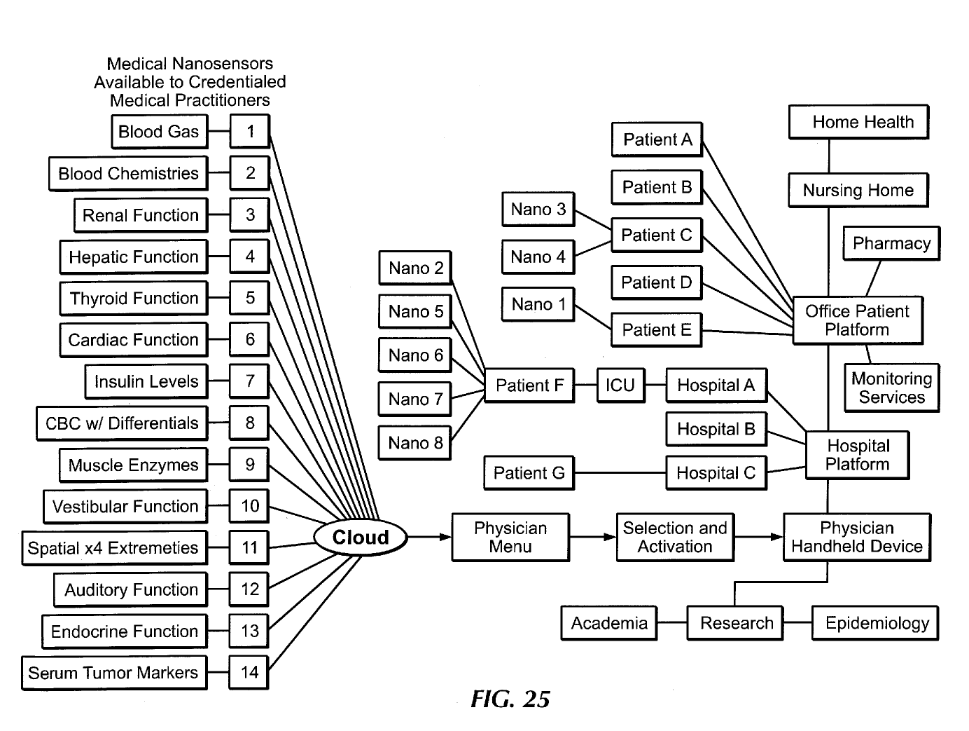

FIG. 25 shows the dMEMs from the perspective of the active practice physician, under an embodiment. Approaching the embodiments described herein from the perspective of the active practice physician, when a new account is opened in his or her name and credential verification has occurred the physician is given direct access to appropriate (non-public) medical apps commensurate to his/her specialty and training. He or she will be able to potentially link-in to his patient's existing user site and add medically monitored sensors that extend beyond normal public access.

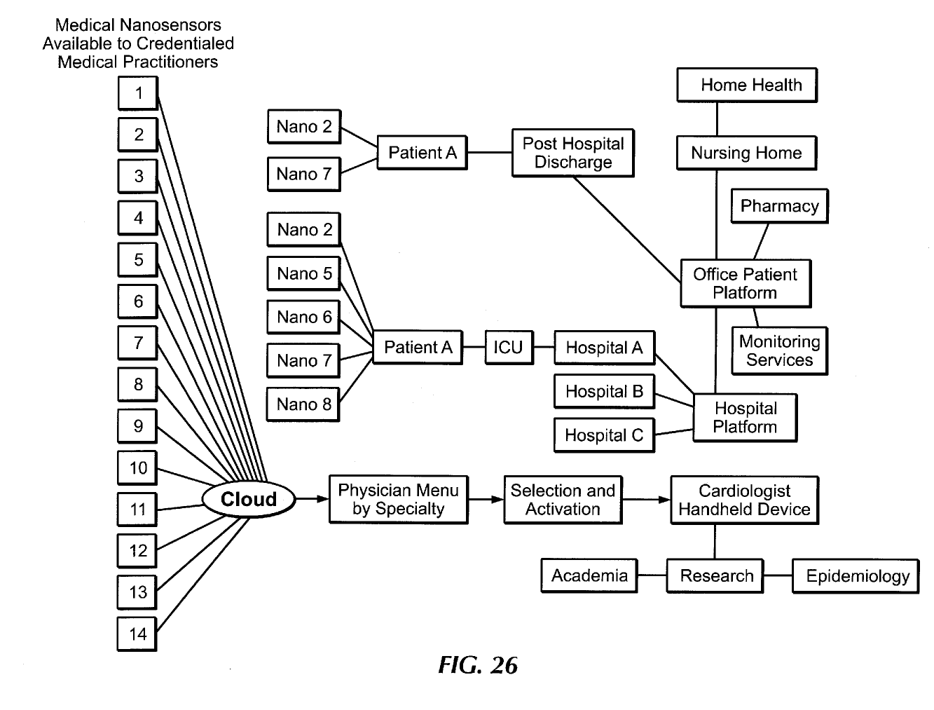

This, for example, may occur upon hospital discharge of a known patient who had been hospitalized for two weeks in acute congestive heart failure. FIG. 26 shows an example of the dMEMs from the perspective of the active practice physician when treating a patient following patient discharge, under an embodiment. The treating cardiologist, in this case, upon patient discharge may wish to continue to follow real-time heart indices post-discharge for two to three weeks. By extending real-time monitoring beyond the hospital stay to the treating physicians handheld or tablet device (perhaps even professional monitoring services), daily medication changes as may become needed would negate what would surely become a hospital re-admission for a similar non-monitored patient.

In another example, a 33-year old female patient is transferred from the Emergency Department to the Critical Care Unit after initial assessment indicates the patient has sustained multi-trauma from a motor vehicle accident one hour earlier. CT Scans on admit to the ER reveal no intra-abdominal or intra-cerebral bleeds, but renal, splenic and hepatic contusions are suspected. The patient has multiple rib fractures, and is breathing on her own and semi-comatose. The admitting Critical Care physician has been apprised of the patient's condition after reading the patient's electronic chart from the emergency department while the patient is in transit to the Critical Care Unit. Upon arrival he performs a complete physical exam. At that juncture, the physician notes the patient has already been identified and has been logged into Hospital's Cloud Port on the 3D Cloud platform.

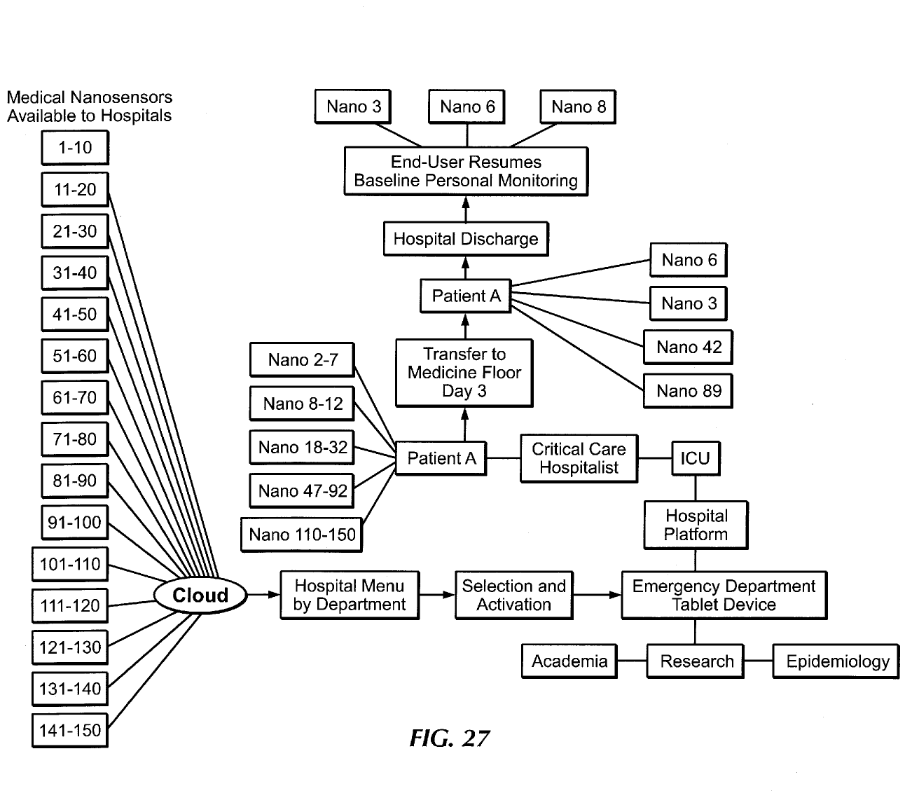

From that point the physician determine what body systems are of most immediate importance to monitor. He will have a handheld tablet with a selection list of medical systems categorized app icons to choose from. The list will have hundreds of individual monitors to choose from as well as lists of single app consolidated nano-sensors. He will make his decision promptly and upon touchpad app selection he will be activating the helical cloud system for immediate recording and feedback. As each nano-sensor or consolidated group of nano-sensors is applied, immediate real-time bedside feedback monitoring is initiated from the Cloud. The physician may select upwards of 50 or more nano-sensors to monitor multi-body systems (e.g., real-time hepatic enzyme flows, cardiac enzymes, renal functions, etc.) all in an effort to preemptively monitor for latent contusional blood loss that could preemptively indicate pending catastrophic organ failure. As the patient's medical condition stabilizes and improves over the next 48 hours the numbers of acute admission (50) nano-sensor functions being monitored may be gradually pruned as condition allows. FIG. 27 shows an example of the dMEMs from the perspective of the active practice physician when treating critical care patient, under an embodiment. The above are just a few cited examples, and in no way are an indication of all potential systems users.

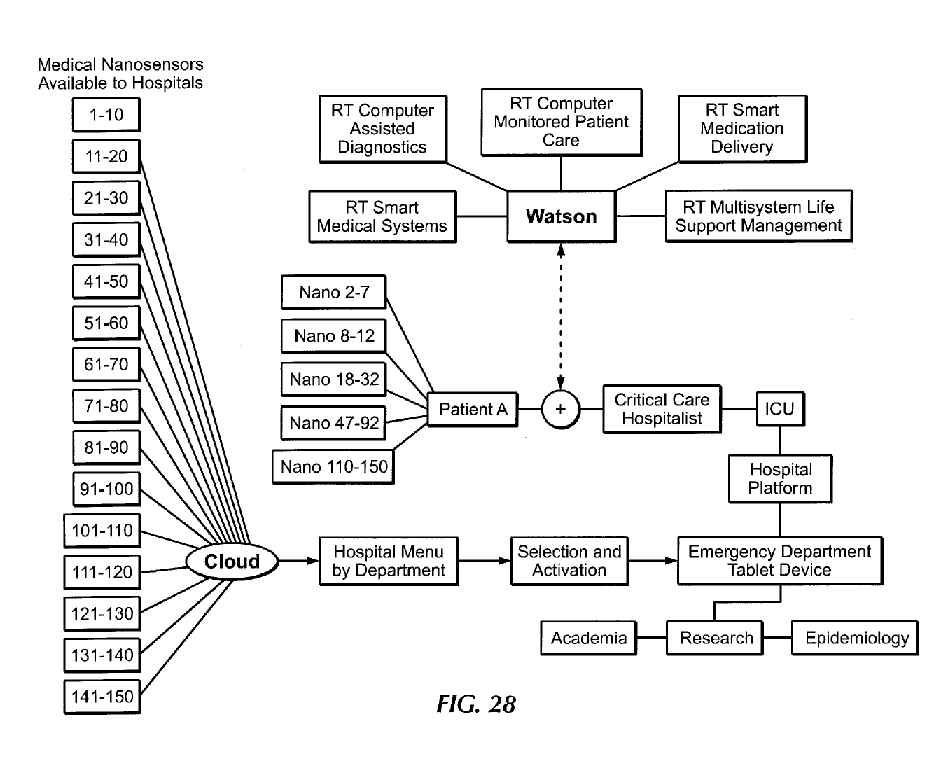

In the above case presentation the medically-necessary selections of nano-sensors were made by the attending acute care physician but embodiments are not so limited. It is promptly anticipated that near future joint venture projects with potential medical smart systems such as IBM's Watson, or comparable system, may allow for the integration of computer assisted diagnosing, as well as computer monitored patient care with eventual real-time computer to patient monitored management for multiple systems life support including medication delivery. FIG. 28 is a block diagram of the dMEM integrated with medical smart systems, under an embodiment.

Embodiments described herein include a method comprising receiving physiological data that includes data of a plurality of physiological parameters collected from an individual entity. The method comprises generating a plurality of micro plots. Each micro plot comprises a cyclical plot of the physiological data for a corresponding time period. Each micro plot corresponds to a time period of a plurality of time periods. The method comprises generating a medical model plot comprising the plurality of micro plots. The plurality of micro plots is plotted chronologically according to the plurality of time periods. A location of an endpoint of each micro plot determines a change in slope of the medical model plot. The slope represents a state of health of the individual entity.

Embodiments described herein include a method comprising: receiving physiological data that includes data of a plurality of physiological parameters collected from an individual entity; generating a plurality of micro plots, wherein each micro plot comprises a cyclical plot of the physiological data for a corresponding time period, wherein each micro plot corresponds to a time period of a plurality of time periods; and generating a medical model plot comprising the plurality of micro plots, wherein the plurality of micro plots are plotted chronologically according to the plurality of time periods, wherein a location of an endpoint of each micro plot determines a change in slope of the medical model plot, wherein the slope represents a state of health of the individual entity.

The physiological data is collected in real-time from sensors coupled to the individual entity.

The sensors comprise nano-sensors.

The sensors comprise sensors coupled to the individual entity.

The sensors comprise sensors implanted in the individual entity.

The method comprises continuously collecting the physiological data.

The physiological data comprises time data.

The physiological data comprises location data.

The physiological data comprises physical activity data.

The time period of the cyclical plot is a 24-hour period.

The cyclical plot is based on a circadian cycle.

The micro plot for each time period comprises a start point and the endpoint.

The endpoint of each micro plot is located at a same point in a complete rotation that defines the micro plot.

The endpoint of each micro plot for each time period is located at a new position in space.

The physiological data determines the new position of the endpoint.

The endpoint of a micro plot is a start point for a next subsequent micro plot.

The medical model plot comprises a continuous helix comprising the plurality of micro plots.

The method comprises compressing the data of the plurality of micro plots to form the medical model plot.

The method comprises determining the state of health by comparing at least one set of micro plots of the medical model plot.

Changes in the slope indicate physical changes in the state of health of the individual entity.

The slope of the medical model plot is inversely proportional to a quality of life of the individual entity.

The slope of the medical model plot represents longevity of the individual entity.

The medical model plot comprises a start point that corresponds to birth of the individual entity.

The medical model plot comprises a normal zone, wherein the normal zone represents absence of disease process in the individual entity.

The medical model plot comprises a subclinical zone, wherein the subclinical zone represents onset of clinical symptoms in the individual entity.

The medical model plot comprises a clinical zone, wherein the clinical zone represents presence of clinical symptoms in the individual entity.

The medical model plot comprises an endpoint that corresponds to death of the individual entity.

The method comprises providing the medical model plot to the individual entity.

The method comprises providing the medical model plot to at least one healthcare provider.

The method comprises providing the medical model plot to at least one organization.

Embodiments described herein include a system comprising a plurality of sensors coupled to an individual entity. The plurality of sensors collect physiological data that includes data of a plurality of physiological parameters collected from the individual entity. The system includes a platform comprising a processor. The platform is coupled to the plurality of sensors. The processor is running an application, and the application generates a plurality of micro plots. Each micro plot comprises a cyclical plot of the physiological data for a corresponding time period. Each micro plot corresponds to a time period of a plurality of time periods. The application generates a medical model plot comprising the plurality of micro plots. The plurality of micro plots is plotted chronologically according to the plurality of time periods. A location of an endpoint of each micro plot determines a change in slope of the medical model plot. The slope represents a state of health of the individual entity.

Embodiments described herein include a system comprising: a plurality of sensors coupled to an individual entity, wherein the plurality of sensors collect physiological data that includes data of a plurality of physiological parameters collected from the individual entity; and a platform comprising a processor, wherein the platform is coupled to the plurality of sensors, wherein the processor is running an application, wherein the application generates a plurality of micro plots, wherein each micro plot comprises a cyclical plot of the physiological data for a corresponding time period, wherein each micro plot corresponds to a time period of a plurality of time periods, wherein the application generates a medical model plot comprising the plurality of micro plots, wherein the plurality of micro plots are plotted chronologically according to the plurality of time periods, wherein a location of an endpoint of each micro plot determines a change in slope of the medical model plot, wherein the slope represents a state of health of the individual entity.

The physiological data is collected in real-time from the plurality of sensors.

The sensors comprise nano-sensors.

The sensors comprise sensors coupled to the individual entity.

The sensors comprise sensors implanted in the individual entity.

The system comprises continuously collecting the physiological data.

The physiological data comprises time data.

The physiological data comprises location data.

The physiological data comprises physical activity data.

The time period of the cyclical plot is a 24-hour period.

The cyclical plot is based on a circadian cycle.

The micro plot for each time period comprises a start point and the endpoint.

The endpoint of each micro plot is located at a same point in a complete rotation that defines the micro plot.

The endpoint of each micro plot for each time period is located at a new position in space.

The physiological data determines the new position of the endpoint.

The endpoint of a micro plot is a start point for a next subsequent micro plot.

The medical model plot comprises a continuous helix comprising the plurality of micro plots.

The data of the plurality of micro plots is compressed, and the medical model plot comprises the compressed data.

The state of health by is determined by comparing at least one set of micro plots of the medical model plot.

The change in the slope corresponds to physical changes in the state of health of the individual entity.

The slope of the medical model plot is inversely proportional to a quality of life of the individual entity.

The slope of the medical model plot corresponds to longevity of the individual entity.

The medical model plot comprises a start point that corresponds to birth of the individual entity.

The medical model plot comprises a normal zone, wherein the normal zone represents absence of disease process in the individual entity.

The medical model plot comprises a subclinical zone, wherein the subclinical zone represents onset of clinical symptoms in the individual entity.

The medical model plot comprises a clinical zone, wherein the clinical zone represents presence of clinical symptoms in the individual entity.

The medical model plot comprises an endpoint that corresponds to death of the individual entity.

The medical model plot is provided to the individual entity.

The medical model plot is provided to at least one healthcare provider.

The medical model plot is provided to at least one organization.

Computer systems and networks suitable for use with the dMEM embodiments described herein include local area networks (LAN), wide area networks (WAN), Internet, or other connection services and network variations such as the world wide web, the public internet, a private internet, a private computer network, a public network, a mobile network, a cellular network, a value-added network, and the like. Computing devices coupled or connected to the network as a component of progressive mechanical intelligence embodiments may be any microprocessor controlled device that permits access to the network, including terminal devices, such as personal computers, workstations, servers, mini computers, main-frame computers, laptop computers, mobile computers, palm top computers, hand held computers, mobile phones, TV set-top boxes, or combinations thereof. The computer network may include one of more LANs, WANs, Internets, and computers. The computers may serve as servers, clients, or a combination thereof.

The dMEM can be a component of a single system, multiple systems, and/or geographically separate systems. The dMEM can also be a subcomponent or subsystem of a single system, multiple systems, and/or geographically separate systems. The dMEM can be coupled to one or more other components (not shown) of a host system or a system coupled to the host system.

One or more components of the dMEM and/or a corresponding system or application to which the dMEM is coupled or connected includes and/or runs under and/or in association with a processing system. The processing system includes any collection of processor-based devices or computing devices operating together, or components of processing systems or devices, as is known in the art. For example, the processing system can include one or more of a portable computer, portable communication device operating in a communication network, and/or a network server. The portable computer can be any of a number and/or combination of devices selected from among personal computers, personal digital assistants, portable computing devices, and portable communication devices, but is not so limited. The processing system can include components within a larger computer system.

The processing system of an embodiment includes at least one processor and at least one memory device or subsystem. The processing system can also include or be coupled to at least one database. The term “processor” as generally used herein refers to any logic processing unit, such as one or more central processing units (CPUs), digital signal processors (DSPs), application-specific integrated circuits (ASIC), etc. The processor and memory can be monolithically integrated onto a single chip, distributed among a number of chips or components, and/or provided by some combination of algorithms. The methods described herein can be implemented in one or more of software algorithm(s), programs, firmware, hardware, components, circuitry, in any combination.

The components of any system that includes the dMEM can be located together or in separate locations. Communication paths couple the components and include any medium for communicating or transferring files among the components. The communication paths include wireless connections, wired connections, and hybrid wireless/wired connections. The communication paths also include couplings or connections to networks including local area networks (LANs), metropolitan area networks (MANS), wide area networks (WANs), proprietary networks, interoffice or backend networks, and the Internet. Furthermore, the communication paths include removable fixed mediums like floppy disks, hard disk drives, and CD-ROM disks, as well as flash RAM, Universal Serial Bus (USB) connections, RS-232 connections, telephone lines, buses, and electronic mail messages.

Unless the context clearly requires otherwise, throughout the description and the claims, the words “comprise,” “comprising,” and the like are to be construed in an inclusive sense as opposed to an exclusive or exhaustive sense; that is to say, in a sense of “including, but not limited to.” Words using the singular or plural number also include the plural or singular number respectively. Additionally, the words “herein,” “hereunder,” “above,” “below,” and words of similar import, when used in this application, refer to this application as a whole and not to any particular portions of this application. When the word “or” is used in reference to a list of two or more items, that word covers all of the following interpretations of the word: any of the items in the list, all of the items in the list and any combination of the items in the list.

The above description of embodiments of the dMEM and corresponding systems and methods is not intended to be exhaustive or to limit the systems and methods to the precise forms disclosed. While specific embodiments of, and examples for, the dMEM and corresponding systems and methods are described herein for illustrative purposes, various equivalent modifications are possible within the scope of the systems and methods, as those skilled in the relevant art will recognize. The teachings of the dMEM and corresponding systems and methods provided herein can be applied to other systems and methods, not only for the systems and methods described above. The elements and acts of the various embodiments described above can be combined to provide further embodiments. These and other changes can be made to the dMEM and corresponding systems and methods in light of the above detailed description.

What is claimed is:

1. A method comprising:
receiving physiological data that includes data of a plurality of physiological parameters collected from an individual entity;
generating a plurality of micro plots, wherein each micro plot comprises a cyclical plot of the physiological data for a corresponding time period, wherein each micro plot corresponds to a time period of a plurality of time periods; and
generating a medical model plot comprising the plurality of micro plots, wherein the plurality of micro plots is plotted chronologically according to the plurality of time periods, wherein a location of an endpoint of each micro plot determines a change in slope of the medical model plot, wherein the slope represents a state of health of the individual entity.

2. The method of claim 1, wherein the physiological data is collected in real-time from sensors coupled to the individual entity.

3. The method of claim 2, wherein the sensors comprise nano-sensors.

4. The method of claim 2, wherein the sensors comprise sensors coupled to the individual entity.

5. The method of claim 2, wherein the sensors comprise sensors implanted in the individual entity.

6. The method of claim 2, comprising continuously collecting the physiological data.

7. The method of claim 1, wherein the physiological data comprises time data.

8. The method of claim 1, wherein the physiological data comprises location data.

9. The method of claim 1, wherein the physiological data comprises physical activity data.

10. The method of claim 1, wherein the time period of the cyclical plot is a 24-hour period.

11. The method of claim 1, wherein the cyclical plot is based on a circadian cycle.

12. The method of claim 1, wherein the micro plot for each time period comprises a start point and the endpoint.

13. The method of claim 12, wherein the endpoint of each micro plot is located at a same point in a complete rotation that defines the micro plot.

14. The method of claim 12, wherein the endpoint of each micro plot for each time period is located at a new position in space.

15. The method of claim 14, wherein the physiological data determines the new position of the endpoint.

16. The method of claim 15, wherein the endpoint of a micro plot is a start point for a next subsequent micro plot.

17. The method of claim 16, wherein the medical model plot comprises a continuous helix comprising the plurality of micro plots.

18. The method of claim 17, comprising compressing the data of the plurality of micro plots to form the medical model plot.

19. The method of claim 17, comprising determining the state of health by comparing at least one set of micro plots of the medical model plot.

20. The method of claim 1, wherein changes in the slope indicate physical changes in the state of health of the individual entity.

21. The method of claim 1, wherein the slope of the medical model plot is inversely proportional to a quality of life of the individual entity.

22. The method of claim 1, wherein the slope of the medical model plot represents longevity of the individual entity.

23. The method of claim 1, wherein the medical model plot comprises a start point that corresponds to birth of the individual entity.

24. The method of claim 23, wherein the medical model plot comprises a normal zone, wherein the normal zone represents absence of disease process in the individual entity.

25. The method of claim 24, wherein the medical model plot comprises a subclinical zone, wherein the subclinical zone represents onset of clinical symptoms in the individual entity.

26. The method of claim 25, wherein the medical model plot comprises a clinical zone, wherein the clinical zone represents presence of clinical symptoms in the individual entity.

27. The method of claim 1, wherein the medical model plot comprises an endpoint that corresponds to death of the individual entity.

28. The method of claim 1, comprising providing the medical model plot to the individual entity.

29. The method of claim 1, comprising providing the medical model plot to at least one healthcare provider.

30. The method of claim 1, comprising providing the medical model plot to at least one organization.

31. A system comprising:
a plurality of sensors coupled to an individual entity, wherein the plurality of sensors collect physiological data that includes data of a plurality of physiological parameters collected from the individual entity; and
a platform comprising a processor, wherein the platform is coupled to the plurality of sensors, wherein the processor is running an application, wherein the application generates a plurality of micro plots, wherein each micro plot comprises a cyclical plot of the physiological data for a corresponding time period, wherein each micro plot corresponds to a time period of a plurality of time periods, wherein the application generates a medical model plot comprising the plurality of micro plots, wherein the plurality of micro plots is plotted chronologically according to the plurality of time periods, wherein a location of an endpoint of each micro plot determines a change in slope of the medical model plot, wherein the slope represents a state of health of the individual entity.

32. The system of claim 31, wherein the physiological data is collected in real-time from the plurality of sensors.

33. The system of claim 32, wherein the sensors comprise nano-sensors.

34. The system of claim 32, wherein the sensors comprise sensors coupled to the individual entity.

35. The system of claim 32, wherein the sensors comprise sensors implanted in the individual entity.

36. The system of claim 32, comprising continuously collecting the physiological data.

37. The system of claim 31, wherein the physiological data comprises time data.

38. The system of claim 31, wherein the physiological data comprises location data.

39. The system of claim 31, wherein the physiological data comprises physical activity data.

40. The system of claim 31, wherein the time period of the cyclical plot is a 24-hour period.

41. The system of claim 31, wherein the cyclical plot is based on a circadian cycle.

42. The system of claim 31, wherein the micro plot for each time period comprises a start point and the endpoint.

43. The system of claim 42, wherein the endpoint of each micro plot is located at a same point in a complete rotation that defines the micro plot.

44. The system of claim 42, wherein the endpoint of each micro plot for each time period is located at a new position in space.

45. The system of claim 44, wherein the physiological data determines the new position of the endpoint.

46. The system of claim 45, wherein the endpoint of a micro plot is a start point for a next subsequent micro plot.

47. The system of claim 46, wherein the medical model plot comprises a continuous helix comprising the plurality of micro plots.

48. The system of claim 47, wherein the data of the plurality of micro plots is compressed, and the medical model plot comprises the compressed data.

49. The system of claim 47, wherein the state of health by is determined by comparing at least one set of micro plots of the medical model plot.

50. The system of claim 31, wherein the change in the slope corresponds to physical changes in the state of health of the individual entity.

51. The system of claim 31, wherein the slope of the medical model plot is inversely proportional to a quality of life of the individual entity.

52. The system of claim 31, wherein the slope of the medical model plot corresponds to longevity of the individual entity.

53. The system of claim 31, wherein the medical model plot comprises a start point that corresponds to birth of the individual entity.

54. The system of claim 53, wherein the medical model plot comprises a normal zone, wherein the normal zone represents absence of disease process in the individual entity.

55. The system of claim 54, wherein the medical model plot comprises a subclinical zone, wherein the subclinical zone represents onset of clinical symptoms in the individual entity.

56. The system of claim 55, wherein the medical model plot comprises a clinical zone, wherein the clinical zone represents presence of clinical symptoms in the individual entity.

57. The system of claim 31, wherein the medical model plot comprises an endpoint that corresponds to death of the individual entity.

58. The system of claim 31, wherein the medical model plot is provided to the individual entity.

59. The system of claim 31, wherein the medical model plot is provided to at least one healthcare provider.

60. The system of claim 31, wherein the medical model plot is provided to at least one organization.
